SEQUENCE LISTING (3 sequences)

SEQ ID NO: 1 - protein, 29 aa, Artificial Sequence
  Description of Artificial Sequence Synthetic peptide
  misc_feature (3)..(3): Xaa can be any naturally occurring amino acid
GLXAAGGDMGNEIDTQNRQIDRIMEKADK

SEQ ID NO: 2 - protein, 7 aa, Artificial Sequence
  Description of Artificial Sequence Synthetic peptide
  misc_feature (3)..(3): Xaa can be any naturally occurring amino acid
GLXAAGG

SEQ ID NO: 3 - protein, 22 aa, Artificial Sequence
  Description of Artificial Sequence Synthetic peptide
DMGNEIDTQNRQIDRIMEKADK

(19) United States
(12) Patent Application Publication
(10) Pub. No.: US 2020/0386746 A1
(43) Pub. Date: Dec. 10, 2020

(54) SENSOR FOR DETECTION OF BOTULINUM TOXIN USING SPUN CARBON NANOTUBE SHEET

(71) Applicant: KOREA RESEARCH INSTITUTE OF STANDARDS AND SCIENCE, Daejeon (KR)

(72) Inventors: Seung-Hoon NAHM, Daejeon (KR); Nam-Hee LEE, Daegu (KR); Kwon-Sang RYU, Daejeon (KR); Un-Bong BAEK, Daejeon (KR)

(73) Assignee: KOREA RESEARCH INSTITUTE OF STANDARDS AND SCIENCE, Daejeon (KR)

(21) Appl. No.: 16/473,794

(22) PCT Filed: Oct. 13, 2017
(86) PCT No.: PCT/KR2017/011319

§ 371 Date: Sep. 3, 2019

(30) Foreign Application Priority Data
Dec. 29, 2016 (KR) 10-2016-0182502

(51) Int. Cl. G01N 33/543; C12Q 1/00; C12Q 1/37; C01B 32/174; G01N 27/12

(52) U.S. Cl. CPC G01N 33/5438; C12Q 1/001; C12Q 1/37; C12Y 304/24069; B82Y 40/00; G01N 27/127; G01N 2333/33; C01B 2202/22; C01B 32/174

(57) ABSTRACT

Disclosed is a sensor for the detection of a botulinum toxin using a carbon nanotube sheet, the sensor including carbon nanotubes and a botulinum toxin receptor formed on the carbon nanotubes.

### SEQUENCE LISTING SPECIFIC REFERENCE

This application contains a Sequence Listing submitted via EFS-Web and hereby incorporated by reference in its entirety. The Sequence Listing is named OAM-131NP-SequenceListing_ST25.txt, created on Sep. 25, 2019, and 1,487 bytes in size.

### TECHNICAL FIELD

The present invention relates to a sensor for the detection of botulinum toxin using a spun carbon nanotube sheet, and more particularly to a sensor for detecting botulinum toxin using a spun carbon nanotube sheet and a method of detecting botulinum toxin using the same.

### BACKGROUND ART

Botulinum toxin is the strongest naturally occurring toxic substance known to date. It is usually found in spoiled food or decayed animals and plants, and the lethal dose thereof for human beings is very low.

Also, botulinum toxin is known to be easy to mass-produce, in addition to the strong toxicity thereof, and thus may be utilized as a biochemical weapon usable in bioterrorism.

There are seven types of botulinum toxin, ranging from A to G, depending on the mechanism thereof. Among these, the toxin targeted in the present invention is the type E toxin, which structurally comprises two polypeptide chains, namely a heavy chain that binds to a neurotransmitter receptor and a light chain that cleaves a neurotransmitter protein through hydrolysis.

The detection methods recognized so far are detection methods based on animal experiments and thus have advantages of high selectivity and high detection capability, but take several days for measurement and are expensive because an animal care facility must be provided for experimentation. Hence, in order to develop a sensor for use in a practical application, a sensing method that has a short detection time and high detection capability and is inexpensive is required.

Meanwhile, Korean Patent Application Publication No. 10-2016-0110643 (hereinafter, referred to as “Cited Document 1”) discloses a sensor for the detection of botulinum toxin using graphene. In Cited Document 1, expensive graphene is used, and the sensor made of graphene in a powder phase is provided, making it difficult to realize a large-area sensor. Even when a virtual large area of graphene is used, problems related to durability and restorability thereof may occur.

### CITATION LIST

(Patent Document 1) Korean Patent Application Publication No. 10-2016-0110643 (Laid-open date: Sep. 22, 2016)

### DISCLOSURE

### Technical Problem

Accordingly, the present invention has been made keeping in mind the problems encountered in the related art, and an objective of the present invention is to provide a large-area sensor for detecting botulinum toxin using a carbon nanotube sheet having superior mechanical properties.

Another objective of the present invention is to provide a method of detecting botulinum toxin using the sensor for detecting botulinum toxin.

Meanwhile, other objectives not mentioned herein will be further contemplated within the range that may be easily deduced from the following detailed description and effects thereof.

### Technical Solution

In order to accomplish the above objectives, an embodiment of the present invention provides a sensor for the detection of botulinum toxin, the sensor comprising carbon nanotubes and a botulinum toxin receptor formed on the carbon nanotubes.

In the sensor for the detection of botulinum toxin according to the embodiment of the present invention, the sensor for the detection of botulinum toxin may include a carbon nanotube sheet.

In the sensor for the detection of botulinum toxin according to the embodiment of the present invention, electrical resistance may increase upon contact of the botulinum toxin with an end of the botulinum toxin receptor.

In the sensor for the detection of botulinum toxin according to the embodiment of the present invention, the botulinum toxin receptor may be at least one selected from among an antibody, an enzyme, a protein, a peptide, an amino acid, an aptamer, a lipid, a cofactor and a carbohydrate.

In the sensor for the detection of botulinum toxin according to the embodiment of the present invention, the peptide may be a peptide having the amino acid comprising SEQ ID NO: 1.

In the sensor for the detection of botulinum toxin according to the embodiment of the present invention, the peptide having the amino acid comprising SEQ ID NO: 1 may bind to the botulinum toxin.

In the sensor for the detection of botulinum toxin according to the embodiment of the present invention, the 22^{nd} arginine (R) and 23^{rd} isoleucine (I) of the peptide having the amino acid comprising SEQ ID NO: 1 may bind to the botulinum toxin to thus cause hydrolysis.

In the sensor for the detection of botulinum toxin according to the embodiment of the present invention, the antibody may be immunoglobulin G.

In the sensor for the detection of botulinum toxin according to the embodiment of the present invention, the sensor may include a linker, which is formed between the carbon nanotubes and the botulinum toxin receptor and is noncovalently bonded with the carbon nanotubes and is covalently bonded with the botulinum toxin receptor.

In the sensor for the detection of botulinum toxin according to the embodiment of the present invention, the linker may be represented by Chemical Formula 1 below:

X-L-Y [Chemical Formula 1]

[in Chemical Formula 1, X is a pyrene group or graphite, L is (CH₂)ₙ, n ranging from 1 to 4, and Y is a hydroxyl group (—OH)].

In the sensor for the detection of botulinum toxin according to the embodiment of the present invention, the linker may be 1-pyrenebutanoic acid succinimidyl ester.

In the sensor for the detection of botulinum toxin according to the embodiment of the present invention, the carbon nanotube sheet may be a surface-treated carbon nanotube sheet.

In addition, the present invention provides a method of detecting botulinum toxin using the sensor for the detection of botulinum toxin as above.

The method of detecting botulinum toxin according to an embodiment of the present invention may include measuring electrical resistance by bringing the above sensor into contact with a sample including botulinum toxin.

In the method of detecting botulinum toxin according to the embodiment of the present invention, the sample including botulinum toxin may contain a metalloproteinase.

### Advantageous Effects

According to the present invention, a sensor for the detection of botulinum toxin is capable of rapidly detecting type E botulinum toxin usable in bioterrorism.

Also, according to the present invention, the sensor for the detection of botulinum toxin is capable of detecting botulinum toxin in an extremely small amount, namely tens of fM (femtomoles).

Moreover, according to the present invention, the sensor for the detection of botulinum toxin can be provided in the form of a flexible large-area sensor using a carbon nanotube sheet.

Also, in the present invention, spinnable carbon nanotubes are used, thus obviating the process for locating carbon nanotubes at a desired portion, whereby carbon nanotubes or a carbon nanotube sheet can be easily aligned at a desired position.

Meanwhile, even effects not explicitly mentioned here, in addition to the effects described in the following specification, which are expected based on the technical features of the present invention, and potential effects thereof, are to be considered as being set forth in the specification of the present invention.

### DESCRIPTION OF DRAWINGS

FIG. 1 schematically shows a sensor for the detection of botulinum toxin according to an embodiment of the present invention;

FIG. 2 is a photograph showing the carbon nanotube sheet 101 used for the sensor for the detection of botulinum toxin according to an embodiment of the present invention;

FIG. 3 is a TEM image showing the carbon nanotubes used for the sensor for the detection of botulinum toxin according to an embodiment of the present invention;

FIG. 4 sequentially shows a process of manufacturing a sensor for the detection of botulinum toxin according to an embodiment of the present invention;

FIG. 5 is graphs showing changes in electrical conductivity of the sensors for the detection of botulinum toxin manufactured in Examples 1 to 3 of the present invention;

FIG. 6 is a graph showing changes in electrical conductivity of the sensors for the detection of botulinum toxin manufactured in Examples 4 to 6 of the present invention; and

FIG. 7 is a graph showing current-voltage curves depending on the radius of curvature of the sensor for the detection of botulinum toxin manufactured in Example 1.

### MODE FOR INVENTION

Hereinafter, the present invention will be described in detail. The following embodiments and drawings are provided as an example so that those skilled in the art can fully understand the concept of the present invention. Moreover, the technical terms and scientific terms used in the present invention have meanings that are typically understood by those of ordinary skill in the art to which this invention belongs, unless otherwise defined. Furthermore, in the following description and the accompanying drawings, a description of known functions and configurations that may unnecessarily obscure the gist of the present invention will be omitted.

The present inventors have made extensive efforts to develop a sensor for detecting a very small amount of botulinum toxin, and have thus ascertained that a sensor for the detection of botulinum toxin, manufactured by subjecting carbon nanotubes to surface treatment or surface modification and binding the carbon nanotubes to a botulinum toxin receptor, is capable of detecting botulinum toxin in the range of fM (femtomoles) at maximum, exhibiting superior mechanical properties such as durability and restorability, and being applicable as a large-area detection sensor, thus culminating in the present invention.

According to the present invention, the sensor for the detection of botulinum toxin includes carbon nanotubes and a botulinum toxin receptor formed on the carbon nanotubes, and a carbon nanotube sheet made of the carbon nanotubes is used.

In the present invention, the term “botulinum toxin receptor” refers to a receptor that is able to bind to the surface of botulinum toxin, and includes at least one selected from among, for example, an antibody, an enzyme, a protein, a peptide, an amino acid, an aptamer, a lipid, a cofactor and a carbohydrate.

As used herein, the term “antibody” refers to an immunoglobulin or an immunoglobulin fragment, including any fragment comprising at least a portion of the variable region of an immunoglobulin molecule that possesses the specific binding force of a full-length immunoglobulin, which is either natural or partially or fully synthesized, for example, recombinantly produced. Thus, an antibody includes any protein having a binding domain that is homologous or substantially homologous to an immunoglobulin antigen-binding domain (antibody-binding site). The antibody may include, but is not limited to, a synthetic antibody, a recombinantly produced antibody, a multispecific antibody, a human antibody, a non-human antibody, a humanized antibody, a chimeric antibody, an intrabody or an antibody fragment.

As used herein, the term “contacting” refers to physical contact or chemical bonding between the botulinum toxin receptor and botulinum toxin. The contact may occur in vitro. For example, a botulinum-toxin-mixed solution may be brought into contact with the sensor for detecting botulinum toxin as described above in a test tube or a container made of a polymer. Here, the botulinum-toxin-mixed solution may be a sample including botulinum toxin, as will be described later.

As used herein, the term “binding” may refer to direct or indirect bonding between two or more media (materials). For example, direct bonding may be chemical bonding between the botulinum toxin receptor and the botulinum toxin, and indirect bonding may mean that the botulinum toxin receptor and the carbon nanotubes bind together with or through a mediator to thus form a complex. In an embodiment of the present invention, the mediator may be a linker.

As used herein, the term “linker” may refer to a connector that is able to connect two different fusion partners (e.g., biological polymers) through hydrogen bonding, electrostatic interaction, van der Waals interaction, disulfide bonding, a salt bridge, hydrophobic interaction, covalent bonding, etc. For example, the linker may be a compound that is able to connect the botulinum toxin receptor and the carbon nanotubes to each other.

As used herein, the term “noncovalent bonding” means a weak interaction for forming an assembly of atoms or molecules by an interaction other than covalent bonding, and examples thereof include interactions such as electrostatic interaction, hydrophobic interaction, hydrogen bonding, and van der Waals interaction.

As also used herein, the term “electrostatic interaction” may refer to a bond that depends on the electrical attraction between ions having opposite charges, the term “hydrophobic interaction” may refer to a bond depending on the tendency of hydrophobic molecules to avoid an interaction with a polar solvent and to realize thermodynamic stabilization, and the term “hydrogen bonding” may refer to a dipole-dipole interaction that occurs between polar covalently bonded molecules formed by bonding hydrogen with fluorine, oxygen, or nitrogen. Furthermore, the term “van der Waals interaction” may refer to a bond resulting from the actions of attraction and repulsion between molecules due to the molecular polarity caused by van der Waals force.

As used herein, the term “spacer” may refer to a peptide sequence or a short amino acid sequence that binds to the linker.

As used herein, the term “surface modification” may refer to varying or altering the surface of a material to be modified in order to prevent changing of the basic properties of the material and to facilitate binding to other materials. For example, surface modification of the carbon nanotubes or carbon nanotube sheet may be performed to achieve binding thereof to the botulinum toxin receptor. The material that enables the surface modification of the carbon nanotubes or the carbon nanotube sheet may be the linker described above.

As used herein, the term “surface treatment” means performing treatment for increasing the energy barrier between the electrode and the carbon nanotubes or between the electrode and the carbon nanotube sheet. Upon surface treatment of the carbon nanotubes or the carbon nanotube sheet, a defect portion may be formed on the surface of the carbon nanotubes or carbon nanotube sheet. Examples of the surface treatment include UV-ozone treatment, gas plasma treatment (where the gas is at least one of hydrogen, methane, and oxygen), and electrical breakdown, and the carbon nanotubes or carbon nanotube sheet may be imparted with p-type semiconductor properties through surface treatment.

As used herein, an “isolated” or “purified” peptide (e.g. an isolated antibody or an antigen-binding fragment thereof) or biologically active portion thereof (e.g. an isolated antigen-binding fragment) is substantially free of cellular material or contaminating proteins from the cell or tissue from which the protein is derived or is substantially free of chemical precursors or other chemicals when chemically synthesized. The formulation may be determined to be free of easily detectable impurities through standard analytical methods that are performed by those skilled in the art to measure purity, such as thin-layer chromatography (TLC), gel electrophoresis and high-performance liquid chromatography (HPLC), or may be determined not to require further purification if the formulation is sufficiently pure so as not to detectably alter the physical and chemical properties of the material, for example, enzymatic and biological activities. Compound purification methods for preparing substantially chemically pure compounds are known to those skilled in the art. However, a substantially chemically pure compound may be a mixture of stereoisomers. In this case, further purification may increase the specific properties of the compound.

As used herein, the term “substrate” may mean an element that functions as a support. Examples of such a substrate include rigid substrates and flexible substrates. Specific examples of rigid substrates include glass substrates including soda lime glass and ceramic substrates such as alumina, etc., and specific examples of flexible substrates include polymer substrates such as polyimide, PDMS (polydimethylsiloxane) and the like. Since the polymer substrate is excellent from the aspects of flexibility, restorability, durability, etc., it is desirable that a polymer substrate be used as a substrate according to the present invention.

Hereinafter, a detailed description will be given of the present invention with reference to the accompanying drawings.

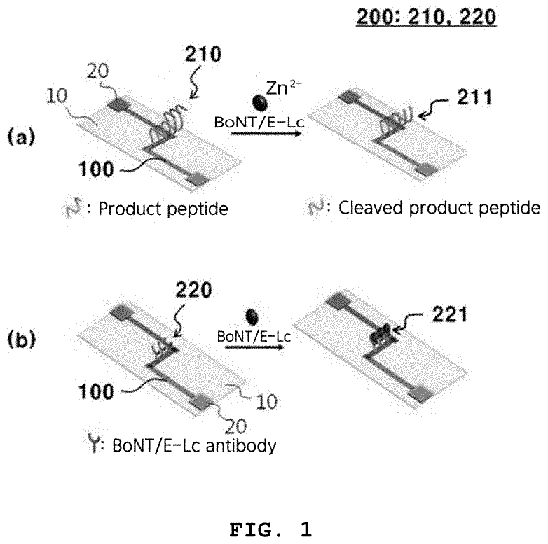

FIG. 1 schematically shows a sensor for the detection of botulinum toxin according to an embodiment of the present invention.

With reference to FIG. 1, the sensor for the detection of botulinum toxin may include a substrate 10, a carbon nanotube sheet 100 formed on the substrate 10, electrodes 20 connected to both ends of the carbon nanotube sheet 100, and a botulinum toxin receptor 200 formed on the carbon nanotube sheet 100.

The botulinum toxin receptor 200 may be at least one selected from among an antibody, an enzyme, a protein, a peptide, an amino acid, an aptamer, a lipid, a cofactor and a carbohydrate.

Specifically, FIG. 1(a) schematically shows a sensor for the detection of botulinum toxin when a peptide 210 is used as the botulinum toxin receptor 200. As shown in FIG. 1(a), the sensor for the detection of botulinum toxin according to an embodiment of the present invention may be configured such that the peptide 210 is formed on the carbon nanotube sheet 100. Here, the peptide 210 may be a peptide having the amino acid comprising SEQ ID NO: 1.

With reference to FIG. 1(a), when botulinum toxin (BonT/E-Lc) comes into contact with one end of the peptide 210, the 22^{nd} arginine (R) and 23^{rd} isoleucine (I) of the peptide having the amino acid comprising SEQ ID NO: 1 bind to the botulinum toxin.

Here, the 22^{nd} arginine (R) and the 23^{rd} isoleucine (I) may bind to the botulinum toxin to thus cause hydrolysis. After the hydrolysis, an isolated peptide 211 may be formed on the carbon nanotube sheet 100. Here, the botulinum toxin may be used in combination with a sample containing metalloproteinase. When using the sample containing metalloproteinase, the sensor for the detection of botulinum toxin according to the present invention is improved in sensitivity of detection of the botulinum toxin. For example, the metalloproteinase may contain ions such as Mn²⁺, Zn²⁺, Ba²⁺, Cu²⁺, Co²⁺, Ca²⁺, Mg²⁺, Ni²⁺, Fe²⁺ and the like. In an embodiment of the present invention, the metalloproteinase may be used in an amount of 0.1 to 80 moles relative to 1 mole of peptide. Given the above molar amount, the metalloproteinase is able to shorten the reaction time upon detection of botulinum toxin and to further increase the capability of detection of botulinum toxin.

Meanwhile, a spacer having the amino acid comprising SEQ ID NO: 2 may have a spiral structure, and may be covalently bonded with the N terminal having the amino acid comprising SEQ ID NO: 1 through the linker, as will be described later.

FIG. 1(b) schematically shows a sensor for the detection of botulinum toxin when an antibody 220 is used as the botulinum toxin receptor 200. As shown in FIG. 1(b), the sensor for the detection of botulinum toxin according to an embodiment of the present invention may be configured such that the antibody 220 is formed on the carbon nanotube sheet 100 in order to make use of the antigen-antibody reaction.

With reference to FIG. 1(b), when botulinum toxin (BonT/E-Lc) comes into contact with one side of the antibody 220, the botulinum toxin is seated on the antibody 220 to thus form a structure 221 in which the botulinum toxin and the antibody 200 bind to each other.

Here, the antibody 220 may be immunoglobulin G. When immunoglobulin G is used as the antibody, the sensor for the detection of botulinum toxin according to the present invention is able to detect botulinum toxin in the range of fM (femtomoles) at maximum.

The antibody 220 may be covalently bonded with the linker, as will be described later.

Although not shown in FIG. 1, the sensor for the detection of botulinum toxin according to the present invention may include the linker formed between the carbon nanotubes of the carbon nanotube sheet 100 and the botulinum toxin receptor 200.

The linker may be noncovalently bonded with the carbon nanotubes 100, and may be covalently bonded with the botulinum toxin receptor 200.

In the sensor for the detection of botulinum toxin according to an embodiment of the present invention, the linker may be represented by Chemical Formula 1 below:

X-L-Y [Chemical Formula 1]

[in Chemical Formula 1, X is a pyrene group or graphite, L is (CH₂)ₙ, n ranging from 1 to 4, and Y is a hydroxyl group (—OH)].

Specifically, the linker may be 1-pyrenebutanoic acid succinimidyl ester.

In an embodiment of the present invention, the linker may be used in an amount of 100 to 10000 moles relative to 1 mole of the botulinum toxin receptor.

When the linker is used in an amount less than 100 moles or exceeding 10000 moles relative to 1 mole of the toxin receptor, the amount of the botulinum toxin binding to the receptor may decrease. Specifically, if the linker is used in an amount less than 100 moles relative to 1 mole of the toxin receptor, the number of receptors binding to the linker may decrease, undesirably lowering the capability of detection of botulinum toxin. On the other hand, if the linker is used in an amount exceeding 10000 moles relative to 1 mole of the toxin receptor, linkers may aggregate on the surface of the carbon nanotubes, and thus the number of receptors binding to the linker may decrease, undesirably lowering the capability of detection of botulinum toxin.

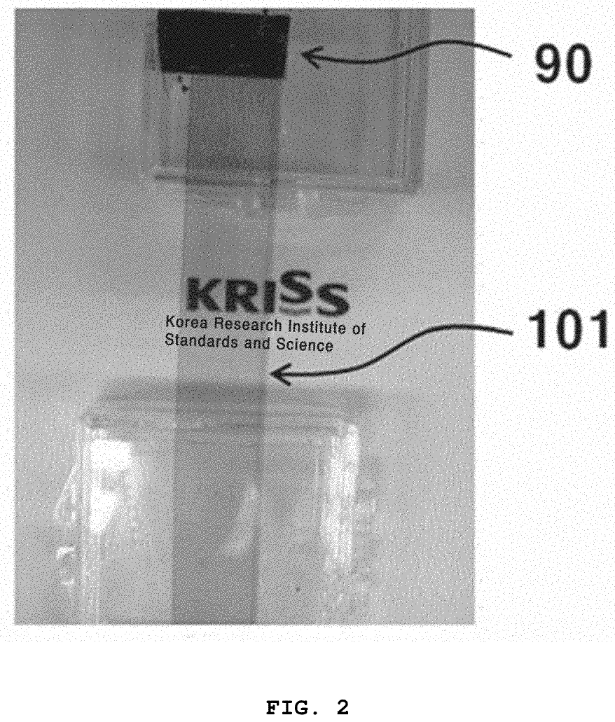

FIG. 2 is a photograph showing the carbon nanotube sheet 101 used in the sensor for the detection of botulinum toxin according to an embodiment of the present invention. As shown in FIG. 2, the carbon nanotube sheet 101 may be a spun sheet formed from a carbon nanotube forest 90.

Specifically, the carbon nanotube sheet 101 according to an embodiment of the present invention may be manufactured in the form of a spun sheet by unidirectionally drawing one end of the carbon nanotube forest 90.

The width of the carbon nanotube sheet 101 may be about 0.1 to 100 mm, but the present invention is not limited as to the width of the carbon nanotube sheet 101. When the carbon nanotube sheet 101 having the above width is used, the sensor for the detection of botulinum toxin according to the present invention may be applied as a large-area sensor.

Also, the carbon nanotube sheet according to an embodiment of the present invention may be a surface-treated carbon nanotube sheet. Thus, the electrical conductivity of the carbon nanotube sheet according to the present invention may be 1 to 3 pS. If the sensor for the detection of botulinum toxin in which the electrical resistance of the carbon nanotube sheet is less than 1 pS is used, it is difficult to detect a botulinum toxin signal of tens of MΩ or more. On the other hand, if the sensor for the detection of botulinum toxin in which the electrical resistance of the carbon nanotube sheet exceeds 3 pS is used, changes in electrical conductivity for an extremely small amount of botulinum toxin are very small, undesirably lowering reliability.

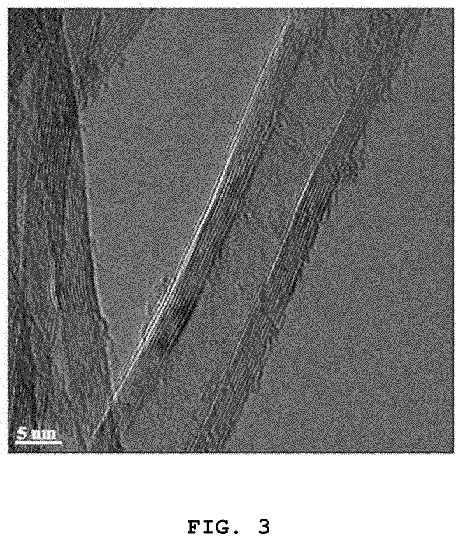

FIG. 3 is a TEM image showing the carbon nanotubes used in the sensor for the detection of botulinum toxin according to an embodiment of the present invention. As described above, the carbon nanotube sheet 100, 101 may be composed of the carbon nanotubes. With reference to FIG. 3, the carbon nanotubes according to an embodiment of the present invention may be multi-walled carbon nanotubes.

In addition, the present invention pertains to a method of detecting botulinum toxin using the aforementioned sensor for the detection of botulinum toxin.

According to the present invention, the method of detecting botulinum toxin includes measuring electrical resistance by bringing the sensor for the detection of botulinum toxin into contact with a sample including botulinum toxin.

Here, the sample including botulinum toxin may contain the metalloproteinase as above.

A better understanding of the present invention will be given through the following preparation example and examples, which are not to be construed as limiting the present invention.

### Preparation Example 1: Manufacture of Carbon Nanotube Sheet

As catalyst layers, an aluminum (Al) layer and an iron (Fe) layer were deposited through physical vapor deposition (PVD) on a silicon substrate comprising a SiO₂ film having a thickness of 500 nm. Here, the aluminum layer had a thickness of 7 nm, and the thickness of the iron layer was 2 nm. Then, a carbon nanotube forest grown perpendicular to the substrate was manufactured through chemical vapor deposition (CVD). Upon CVD, the deposition temperature was 750□, the pressure was 700 torr, the injection gas was composed of 270 sccm of argon, 450 sccm of hydrogen, and 100 sccm of ethylene, and the deposition holding time was 1 hr.

The carbon nanotubes thus prepared had a diameter of about 5 to 100 nm and a length of about 750 μm. Then, one end of the carbon nanotube forest was unidirectionally drawn at a predetermined angle, thereby manufacturing a spun carbon nanotube sheet having a length of 8 mm and a width of 1 mm.

### Example 1

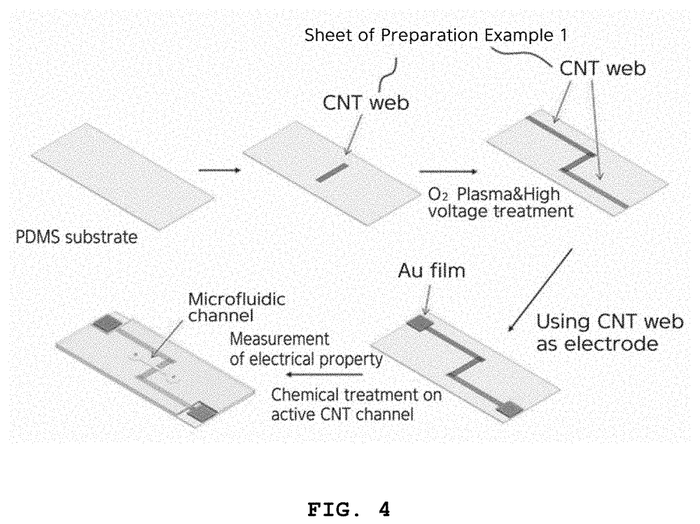

As shown in FIG. 4, the carbon nanotube sheet manufactured in Preparation Example 1 was attached onto a PDMS substrate, oxygen plasma treatment was performed, and a voltage of about 50 V was applied across the ends of the carbon nanotube sheet to thus cause electrical breakdown, thereby manufacturing a surface-treated carbon nanotube sheet. Then, the first carbon nanotube sheet and the second carbon nanotube sheet manufactured in Preparation Example 1 were attached to respective ends of the above carbon nanotube sheet to form electrodes, and one end of each of the first carbon nanotube sheet and the second carbon nanotube sheet spaced apart from the surface-treated carbon nanotube sheet was coated with a gold (Au) electrode.

Thereafter, a 6 mM 1-pyrenebutanoic acid succinimidyl ester linker solution was injected to the surface of the surface-treated carbon nanotube sheet, whereby the linker was bonded thereto. Then, the linker-bonded carbon nanotube sheet was washed with dimethylformamide (DMF) and a buffer solution, and a 0.8 μM peptide solution comprising SEQ ID NO: 1 was injected thereto, thereby manufacturing a sensor for the detection of botulinum toxin, configured such that a monolayer was formed on the surface of the carbon nanotube sheet.

Finally, the channel portion of the carbon nanotube sheet was washed with a buffer solution, and while a sample containing 3 nM botulinum toxin (botulinum toxin type E light chain) was allowed to flow to a fluidic channel across the carbon nanotube sheet, the electrical conductivity of the sensor for the detection of botulinum toxin was measured. Here, the sample contained 20 μM ZnCl₂.

### Example 2

This example was performed in the same manner as in Example 1, with the exception that 0.3 nM botulinum toxin was used.

### Example 3

This example was performed in the same manner as in Example 1, with the exception that 60 pM botulinum toxin was used.

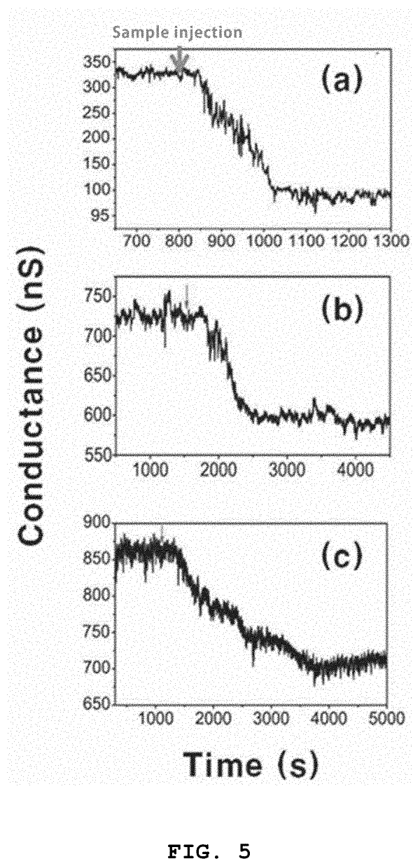

FIG. 5 is graphs showing changes in the electrical conductivity of the sensors for the detection of botulinum toxin manufactured in Examples 1 to 3. Specifically, FIG. 5(a) shows the graph of Example 1, FIG. 5(b) shows the graph of Example 2, and FIG. 5(c) shows the graph of Example 3. As shown in FIG. 5, it is confirmed that the sensor for the detection of botulinum toxin of Examples 1 to 3 was capable of detecting about 10 nM to 10 pM botulinum toxin.

### Example 4

As shown in FIG. 4, the carbon nanotube sheet manufactured in Preparation Example 1 was attached onto a PDMS substrate, oxygen plasma treatment was performed, and a voltage of about 50 V was applied across the ends of the carbon nanotube sheet to thus cause electrical breakdown, thereby manufacturing a surface-treated carbon nanotube sheet. Then, the first carbon nanotube sheet and the second carbon nanotube sheet manufactured in Preparation Example 1 were attached to respective ends of the above carbon nanotube sheet to form electrodes, and one end of each of the first carbon nanotube sheet and the second carbon nanotube sheet spaced apart from the surface-treated carbon nanotube sheet was coated with a gold (Au) electrode.

Thereafter, a 6 mM 1-pyrenebutanoic acid succinimidyl ester linker solution was injected to the surface of the surface-treated carbon nanotube sheet, whereby the linker was bonded thereto. Then, the linker-bonded carbon nanotube sheet was washed with dimethylformamide (DMF) and a buffer solution, and a 0.8 μM peptide solution comprising SEQ ID NO: 1 was injected thereto, thereby manufacturing a sensor for the detection of botulinum toxin, configured such that a monolayer was formed on the surface of the carbon nanotube sheet.

Thereafter, a 6 mM 1-pyrenebutanoic acid succinimidyl ester linker solution was injected to the surface of the surface-treated carbon nanotube sheet, whereby the linker was bonded thereto. Then, the linker-bonded carbon nanotube sheet was washed with dimethylformamide (DMF) and a buffer solution and mixed with 10 μM IgG (made by KAIST), serving as an antibody against BoNT/E, thus forming a monolayer on the surface of the carbon nanotube sheet, thereby manufacturing a sensor for the detection of botulinum toxin.

Finally, the channel portion of the carbon nanotube sheet was washed with a buffer solution, and while a sample containing 51 fM botulinum toxin (botulinum toxin type E light chain) was allowed to flow to a fluidic channel across the carbon nanotube sheet, the electrical conductivity of the sensor for the detection of botulinum toxin was measured.

### Example 5

This example was performed in the same manner as in Example 4, with the exception that 100 fM botulinum toxin was used.

### Example 6

This example was performed in the same manner as in Example 4, with the exception that 500 fM botulinum toxin was used.

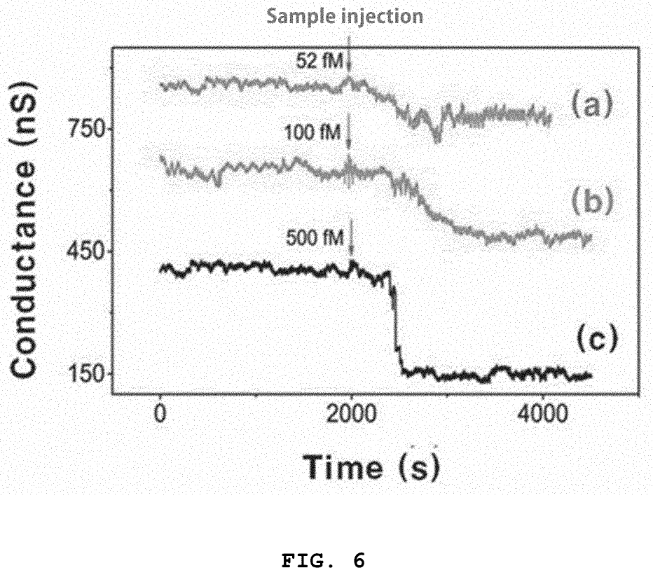

FIG. 6 is a graph showing changes in the electrical conductivity of the sensors for the detection of botulinum toxin manufactured in Examples 4 to 6. In FIG. 6, (a) shows the graph of Example 4, (b) shows the graph of Example 5, and (c) shows the graph of Example 6. As shown in FIG. 6, it can be confirmed that the sensor for the detection of botulinum toxin of Examples 4 to was capable of detecting about 10 fM to 1000 fM botulinum toxin.

Therefore, it can be concluded that the sensor for the detection of botulinum toxin according to the present invention enables accurate measurement even at 1 to 13 ng/kg (about 55 fM) or less, which is the median lethal dose (LD₅₀) of the botulinum toxin for human beings.

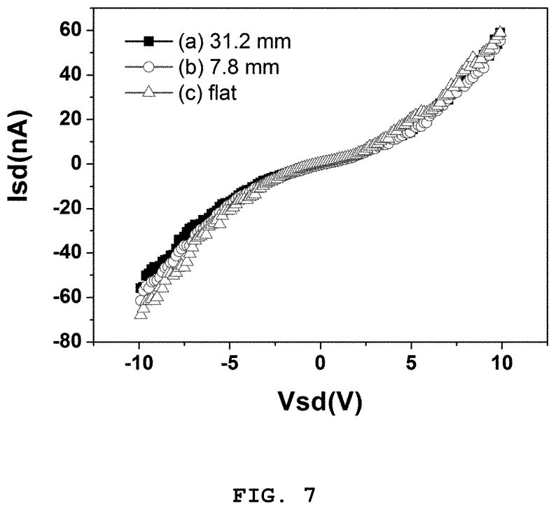

FIG. 7 is a graph showing the current-voltage curves depending on the radius of curvature of the sensor for the detection of botulinum toxin manufactured in Example 1. In FIG. 7, (a) shows a graph in which the radius of curvature of the sensor for the detection of botulinum toxin is 31.2 mm, (b) shows a graph in which the radius of curvature of the sensor for the detection of botulinum toxin is 7.8 mm, and (c) shows a graph in which the sensor for the detection of botulinum toxin is flat. As shown in FIG. 7, even when the radius of curvature in which the sensor for the detection of botulinum toxin is curved was increased, there was almost no change in the current-voltage curve. Therefore, it is considered that the sensor for toxin detection according to the present invention can be applied as a flexible sensor.

As described hereinbefore, the present invention has been described in connection with specified items and predetermined embodiments and drawings, which are merely set forth to provide a better understanding of the present invention, and the present invention is not limited to the above embodiments, from which various changes and modifications are possible, as will be apparent to those skilled in the art.

Accordingly, the spirit of the present invention should not be confined to the disclosed embodiments, and should be defined by all modifications or modified forms derived from the accompanying claims and equivalents thereto.

### DESCRIPTION OF REFERENCE NUMERALS IN THE DRAWINGS

10: substrate 20: electrode 90: carbon nanotube forest
100, 101: carbon nanotube sheet
200: botulinum toxin receptor 210: peptide
220: antibody

1. A sensor for detecting a botulinum toxin using a carbon nanotube sheet, the sensor comprising: carbon nanotubes and a botulinum toxin receptor formed on the carbon nanotubes.

2. The sensor of claim 1, wherein electrical resistance increases upon contact of the botulinum toxin with an end of the botulinum toxin receptor.

3. The sensor of claim 1, wherein the botulinum toxin receptor is at least one selected from among an antibody, an enzyme, a protein, a peptide, an amino acid, an aptamer, a lipid, a cofactor and a carbohydrate.

4. The sensor of claim 3, wherein the peptide is a peptide having an amino acid comprising SEQ ID NO: 1.

5. The sensor of claim 4, wherein 22^{nd} arginine (R) and 23^{rd} isoleucine (I) of the peptide having the amino acid comprising SEQ ID NO: 1 bind to the botulinum toxin to thus cause hydrolysis.

6. The sensor of claim 3, wherein the antibody is immunoglobulin G.

7. The sensor of claim 1, wherein the sensor includes a linker, which is formed between the carbon nanotubes and the botulinum toxin receptor and is noncovalently bonded with the carbon nanotubes and is covalently bonded with the botulinum toxin receptor.

8. The sensor of claim 7, wherein the linker is represented by Chemical Formula 1 below:
X-L-Y [Chemical Formula 1]
[in Chemical Formula 1, X is a pyrene group or graphite, L is (CH₂)ₙ, n ranging from 1 to 4, and Y is a hydroxyl group (—OH)].

9. The sensor of claim 8, wherein the linker is 1-pyrenebutanoic acid succinimidyl ester.

10. The sensor of claim 1, wherein the carbon nanotube sheet is a surface-treated carbon nanotube sheet.

11. A method of detecting botulinum toxin, the method comprising:
measuring electrical resistance by bringing the sensor of claim 1 into contact with a sample including botulinum toxin.

12. The method of claim 11, wherein the sample including botulinum toxin contains a metalloproteinase.
